(19) United States
(12) Patent Application Publication
(10) Pub. No.: US 2023/0017172 A1
(43) Pub. Date: Jan. 19, 2023

(54) SELF-ADAPTIVE MULTI-SCALE RESPIRATORY MONITORING METHOD BASED ON CAMERA

(71) Applicant: Shenzhen iBaby Labs Inc, Shenzhen (CN)

(72) Inventors: Yong XU, Shenzhen (CN); Yulai HUANG, SHENZHEN (CN)

(21) Appl. No.: 17/637,903

(22) PCT Filed: Apr. 30, 2021
(86) PCT No.: PCT/CN2021/091628

§ 371 Date: Feb. 24, 2022

(51) Int. Cl. A61B 5/08

(52) U.S. Cl. CPC A61B 5/0816; A61B 2503/04; G06T 2207/10016

(57) ABSTRACT

A self-adaptive multi-scale respiratory monitoring method based on a camera, relates to the technical field of video image signal identification processing, in order to solve the defect that the local optimal respiratory signal and the global optimal respiratory signal cannot be acquired by single image scale, a method was provided: (1) acquiring a respiratory monitoring object in real time;(2) performing multi-scale regular pre-segmentation on a video image, performing local respiratory signal identification and extraction on each unit area pre-segmented under each scale respectively, and defining the unit area with local respiratory signal output as a target area; and (3) comparing local respiratory signals extracted from the target area pre-segmented under each scale, determining an optimal segmentation scale, and taking a local respiratory signal extracted from the target area under the optimal segmentation scale as a monitoring respiratory signal output. The reliability is improved, and intelligent monitoring is realized.

### FIELD OF TECHNOLOGY

The present disclosure relates to the technical field of video image signal identification processing.

### BACKGROUND

Respiratory frequency is a sensitive index of acute respiratory dysfunction, and is also an important index to measure whether the human heart function is good or bad and the gas exchange is normal. Normal adults breathe about 12-20 times per minute, while children breathe faster than adults, reaching 20-30 times per minute; the respiratory frequency of newborns may reach 44 times per minute; and the ratio of respiration to pulse is 1:4, that is, the pulse beats 4 times at every breath. At present, two basic monitoring methods of the respiratory frequency are: a direct monitoring method and an indirect monitoring method. The direct monitoring method includes an impedance method, a temperature sensor method, a pressure sensor method, a carbon dioxide method, a breath sound method and an ultrasonic method; and the indirect monitoring method includes methods for monitoring the respiratory frequency through electrocardio (ECG), blood pressure, myoelectricity and photoplethysmography.

The method for non-contact monitoring respiration based on a camera has emerged in recent years. Respiratory signals may be monitored without touching the subject’s body, thereby reducing the discomfort and inconvenience caused by wearable devices, improving user experience and simplifying the monitoring process. The respiratory monitoring based on the camera mainly adopts three principles: (1) change of blood volume; (2) change of nasal cavity temperature; and (3) chest/abdominal breathing movement. The mode (3) is more commonly used because of its high reproducibility; however, the respiratory monitoring based on chest/abdominal breathing movement adopts a preset fixed scale according to the image resolution to perform respiratory signal extraction at single image scale, but the single image scale cannot achieve the optimal respiratory signal extraction effect. The main reasons are as follows: (1) the area where the local texture is more obvious needs a smaller image scale to extract the respiratory signal so as to achieve a better sensitivity, and the preset fixed scale is not necessarily the most suitable scale; and (2) the area where the local texture is not obvious needs a larger image scale to extract the signal so as to include more texture information and make the extraction of the breathing movement more accurate. However, the local texture of the respiratory monitoring object is bound to be different, for example: clothing texture and wrinkle, uneven illumination, etc. Therefore, the local optimal respiratory signal and the global optimal respiratory signal cannot be acquired from the single image scale.

### SUMMARY

To sum up, an objective of the present disclosure is to provide a self-adaptive multi-scale respiratory monitoring method based on a camera so as to solve the technical limitation that the local optimal respiratory signal and the global optimal respiratory signal cannot be acquired by single image scale.

To solve the technical problem provided by the present disclosure, the present disclosure proposes the following technical solution:

a self-adaptive multi-scale respiratory monitoring method based on a camera includes the following steps:
(1) acquiring a respiratory monitoring object by the camera in real time;
(2) performing multi-scale regular pre-segmentation on a video image acquired by the camera, performing local respiratory signal identification and extraction on each unit area pre-segmented under each scale respectively, and defining the unit area with local respiratory signal output as a target area; and
(3) comparing local respiratory signals extracted from the target area pre-segmented under each scale, determining an optimal segmentation scale according to the quality of the local respiratory signals, and taking a local respiratory signal extracted from the target area under the optimal segmentation scale as a monitoring respiratory signal output.

The technical solution for further limiting the present disclosure includes:

in the step (3), when more than two target areas under the same scale are present, the local respiratory signals extracted from the two target areas with a highest pixel position coincidence degree under two different scales are compared; and a plurality of local respiratory signals extracted from a plurality of target areas under the optimal segmentation scale are synthesized or compared to obtain an optimal local respiration as a monitoring respiratory signal output.

In the step (2), when a local respiratory signal meeting a preset value cannot be extracted, a pixel of a local area is subjected to single-scale irregular segmentation through the guidance of a local feature of image content to obtain several unit areas with an approximate pixel feature, and each unit area is subjected to local respiratory signal identification and extraction; the unit area with the local respiratory signal output is defined as the target area; and a plurality of local respiratory signals extracted from a plurality of target areas are synthesized or compared to obtain an optimal local respiration as a monitoring respiratory signal output.

The present disclosure has the following beneficial effects: according to the present disclosure, a video image acquired by the camera is subjected to multi-scale regular pre-segmentation, the optimal segmentation scale is adaptively determined according to the quality of the respiratory signal, and the local respiratory signal extracted from the target area under the optimal segmentation scale is taken as the monitoring respiratory signal output, so that the optimal respiratory area and the global optimal respiratory signal under multi-scale are acquired accurately from the respiratory monitoring object monitoring video, the reliability of the camera non-contact monitoring respiratory signal is improved, and intelligent monitoring is realized.

### BRIEF DESCRIPTION OF THE DRAWINGS

FIG. 1 is a work flow chart when the present disclosure adopts a video image to perform multi-scale regular pre-segmentation; and

FIG. 2 is a schematic diagram of single scale irregular segmentation adopted by the present disclosure.

### DESCRIPTION OF THE EMBODIMENTS

The method of the present disclosure is further described below with reference to the accompanying drawings and the preferred specific embodiments of the present disclosure.

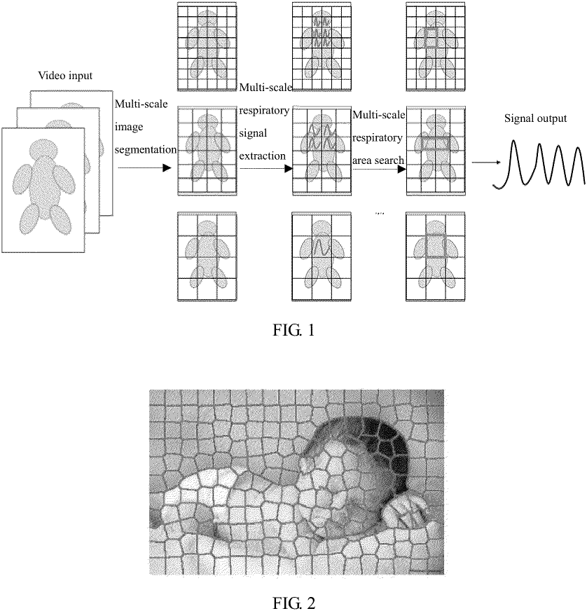

Referring to FIG. 1, a self-adaptive multi-scale respiratory monitoring method based on a camera, disclosed by the present disclosure, includes the following steps:
(1) a respiratory monitoring object is acquired by the camera in real time. Respiratory monitoring objects are mainly children and newborns because children and newborns are immature and not suitable for contact respiratory monitoring; in addition, children and newborns are also high-risk monitoring objects and are high-risk groups with acute respiratory dysfunction. At present, people generally use the camera to perform video and audio acquisition on the crib and perform motion detection on the acquired video to prevent accidents that infants and young children fall out of the bed in their sleep without being guarded because of turning over and crawling and avoid endangering the personal safety of the infants and young children. When the monitored object moves to a great extent, or when crying is identified by the acquired audio, the processor of the camera automatically produces alarm information and short message reminding is conducted by the guardian's smart phone. The camera adopted by the present disclosure may be a color camera which may identify the subtle periodic continuous motion signals of the chest, abdomen, neck or face of the monitored object during breathing on the basis of the motion detection and crying voice identification function of the traditional camera, or may be a monitoring camera with an infrared night vision function.
(2) A video image acquired by the camera is subjected to multi-scale regular pre-segmentation, each unit area pre-segmented under each scale is subjected to local respiratory signal identification and extraction respectively, and the unit area with local respiratory signal output is defined as a target area; and Since the periodic continuous motion amplitude of the chest, abdomen, neck or face of the monitored object during breathing is extremely subtle relative to the global acquired video image, the respiratory frequency can be output efficiently and accurately only when the scale of the target region is suitable. Image segmentation is the common processing process of various kinds of image identification processing at present, and is the process of dividing the image into several unit areas which have feature consistency and do not overlap with each other. The image segmentation of the present disclosure preferably adopts multi-scale regular segmentation, that is, it adopts multiple different scale rules for image segmentation, similar to gridded segmentation, thereby achieving several unit areas. After image segmentation at each time, each unit area is subjected to local respiratory signal identification and extraction respectively. The specific identification and extraction process may include the processing processes such as image graying, histogram equalization, image normalization, video interframe matching, image whitening, removing a strange image to optimize a data set and the like. Generally, if the respiratory monitoring object wears clothes with obvious texture and can be directly acquired by the camera, the unit regions corresponding to the chest and the abdomen will generate obvious periodical continuous motion signals. According to the periodical continuous motion signals, the local respiratory signal may be identified and extracted. The local respiratory signal may be extracted continuously and stably by relatively larger scale regular segmentation. If the respiratory monitoring object wears clothes with single color or the clothes are covered with a quilt with single color, it is difficult for the unit areas corresponding to the chest and the abdomen to identify and extract the respiratory signals, the local respiratory signal only can be extracted from the relatively weak periodical continuous motion signals of the neck or face of the respiratory monitoring object, and relatively small scale regular segmentation only can be adopted. Although the quality of the extracted local respiratory signal is not as good as that of the local respiratory signal extracted by large scale regular segmentation when the texture of the chest and abdomen clothing is obvious, it can at least ensure the local respiratory signal that can be extracted.
(3) comparing local respiratory signals extracted from the target area pre-segmented under each scale, determining an optimal segmentation scale according to the quality of the local respiratory signals, and taking a local respiratory signal extracted from the target area under the optimal segmentation scale as a monitoring respiratory signal output. When more than two target areas under the same scale are present, the local respiratory signals extracted from the two target areas with a highest pixel position coincidence degree under two different scales are compared; a plurality of local respiratory signals extracted from a plurality of target areas under the optimal segmentation scale are compared to obtain an optimal local respiration as a monitoring respiratory signal output; or the plurality of target areas under the optimal segmentation scale are combined as a target area, and a global respiratory signal is comprehensively output. After the local optimal segmentation scale is determined, the segmentation scale parameter setting is used in the subsequent respiratory monitoring. After the content of the respiratory monitoring object changes and the respiratory signal that meets the requirement cannot be identified and extracted under the original segmentation scale, the step (2) is repeated, the optimal segmentation scale parameter is re-determined and the multi-scale respiratory monitoring is adapted.

Since the image content is not considered during multi-scale regular pre-segmentation in the step (2), even if when the local respiratory signal meeting the preset value still cannot be extracted from the relatively weak periodical continuous motion signal of the neck or face of the respiratory monitoring object under the optimal scale. As shown in FIG. 2, a pixel of a local area is subjected to single-scale irregular segmentation through the guidance of the local feature of the image content, several unit areas with an approximate pixel feature are segmented, and each unit area is subjected to local respiratory signal identification and extraction respectively. The unit area with local respiratory signal output is defined as the target area. A plurality of local respiratory signals extracted from the plurality of target areas are synthesized or compared to obtain an optimal local respiration as a monitoring respiratory signal output. The single-scale irregular segmentation is a segmentation method based on mean drift, and color clustering of the feature space is realized through the gradient of the mode space density function, so that the image segmentation is achieved. Segmentation conducted according to the image edge detection is favorable for capturing the relatively weak periodical continuous motion signal of the neck or face.

The present disclosure adopts camera non-contact, multi-scale image segmentation and respiratory signal extraction and searches the optimal respiratory area under multiple scale and the global optimal respiratory signal, thereby realizing respiratory signal monitoring and meeting vital sign guardianship and early warning requirement efficiently and accurately.

What is claimed is:

1. A self-adaptive multi-scale respiratory monitoring method based on a camera, comprising the following steps:
(1) acquiring a respiratory monitoring object by the camera in real time;
(2) performing multi-scale regular pre-segmentation on a video image acquired by the camera, performing local respiratory signal identification and extraction on each unit area pre-segmented under each scale respectively, and defining the unit area with local respiratory signal output as a target area; and
(3) comparing local respiratory signals extracted from the target area pre-segmented under each scale, determining an optimal segmentation scale according to the quality of the local respiratory signals, and taking a local respiratory signal extracted from the target area under the optimal segmentation scale as a monitoring respiratory signal output.

2. The self-adaptive multi-scale respiratory monitoring method based on a camera according to claim 1, wherein in the step (3), when more than two target areas under the same scale are present, the local respiratory signals extracted from the two target areas with a highest pixel position coincidence degree under two different scales are compared; and a plurality of local respiratory signals extracted from a plurality of target areas under the optimal segmentation scale are synthesized or compared to obtain an optimal local respiration as a monitoring respiratory signal output.

3. The self-adaptive multi-scale respiratory monitoring method based on a camera according to claim 1, wherein in the step (2), when a local respiratory signal meeting a preset value cannot be extracted, a pixel of a local area is subjected to single-scale irregular segmentation through the guidance of a local feature of image content to obtain several unit areas with an approximate pixel feature, and each unit area is subjected to local respiratory signal identification and extraction; the unit area with the local respiratory signal output is defined as the target area; and a plurality of local respiratory signals extracted from a plurality of target areas are synthesized or compared to obtain an optimal local respiration as a monitoring respiratory signal output.
